(19) United States
(12) Patent Application Publication
(10) Pub. No.: US 2019/0269934 A1
(43) Pub. Date: Sep. 5, 2019

(54) LIGHT DISPLAY SYSTEM AND METHOD OF TREATMENT USING THE SAME

(71) Applicant: LiteZilla LLC, Cedarburg, WI (US)

(72) Inventor: Adam Butlein, Cedarburg, WI (US)

(21) Appl. No.: 16/289,821

(22) Filed: Mar. 1, 2019

Related U.S. Application Data
(60) Provisional application No. 62/636,972, filed on Mar. 1, 2018.

(51) Int. Cl. A61N 5/06; F21V 3/04; F21V 23/02; A63F 3/00

(52) U.S. Cl. CPC A61N 5/0613; F21V 3/049; F21Y 2115/10; A63F 3/00643; A63F 3/00697; F21V 23/02

(57) ABSTRACT

A light display system including a rear panel, a light source, a diffuser, a front panel, and a power supply. The light source is supported by the rear panel. The light source is configured to output light. The diffuser has a first side and a second side. The first side is proximate the light source. The diffuser is configured to diffuse the light outputted by the light source. The front panel is located proximate the second side of the diffuser. The front panel includes a plurality of apertures, with each aperture configured to receive a peg. The power supply is configured to provide power to the light display.

### RELATED APPLICATIONS

This application claims the benefit to U.S. Provisional Patent Application No. 62/636,972, filed on Mar. 1, 2018, the entire contents of which are incorporated herein by reference.

### FIELD

Embodiments relate to light display system and methods of treating neurological disorders using the same.

### SUMMARY

Light displays may be relatively small (for example, configured to be placed, or set, on a table). Such a sized light display inherently includes relatively small pegs, which may present a choking hazard for small children. Additionally, such relatively small pegs may be easily lost or broken. Thus, a needs exists for a light display system having a size large enough to provide a museum-quality immersion experience, while being able to: (1) withstand the rigor of a high traffic environment; (2) allow children and adults to work together at a single system; and (3) include components, such as pegs, that do not present a choking hazard.

In one embodiment, the application provides a light display system including a rear panel, a light source, a diffuser, a front panel, and a power supply. The light source is supported by the rear panel. The light source is configured to output light. The diffuser has a first side and a second side. The first side is proximate the light source. The diffuser is configured to diffuse the light outputted by the light source. The front panel is located proximate the second side of the diffuser. The front panel includes a plurality of apertures, with each aperture configured to receive a peg. The power supply is configured to provide power to the light display.

In another embodiment the application provides a method of treating a neurological disorder in a subject. The method including providing a light source directed toward the subject and providing a panel between the subject and the light source. The panel includes a plurality of apertures, with each aperture configured to receive a peg placed by the subject for treating the neurological disorder. Wherein the peg is illuminated by the light source when placed in an aperture of the plurality of apertures. In some embodiments, the method further includes varying, via a controller, the light source directed toward the subject for treating the neurological disorder. In some embodiments, the method further includes varying, via a controller, the light source directed toward subject for purposes of team building, brand activations, and as a teaching aid.

Other aspects of the application will become apparent by consideration of the detailed description and accompanying drawings.

### BRIEF DESCRIPTION OF THE DRAWINGS

FIG. 1 is a front perspective view of a light display system according some embodiments.

FIG. 2 is a front view of the light display system of FIG. 1 according to some embodiments.

FIG. 3 is a side view of the light display system of FIG. 1 according to some embodiments.

FIG. 4 is a top view of the light display system of FIG. 1 according to some embodiments.

FIG. 5 is a front perspective view, with a front panel removed, of the light display system of FIG. 1 according to some embodiments.

FIG. 6 is an enlarged, cutaway view of a front panel of the light display system of FIG. 1 according to some embodiments.

FIG. 7 is a side cutaway view of the light display system of FIG. 1 according to some embodiments.

FIGS. 8A & 8B illustrate pegs of the light display system of FIG. 1 according to some embodiments.

FIG. 9 is a block diagram of a control system of the light display system of FIG. 1 according to some embodiments.

FIG. 10 is a mounting device of the light display system of FIG. 1 according to some embodiments.

FIG. 11 is a perspective view of a light display system according to some embodiments.

FIG. 12 is a perspective view of a light display system according to some embodiments.

FIG. 13 is a perspective view of a light display system according to some embodiments.

FIG. 14 is a perspective view of a light display system according to some embodiments.

FIG. 15 is a front perspective view, with a front panel removed, of the light display system of FIG. 1 according to some embodiments.

FIGS. 16A-16C illustrate a cart for supporting a light display system according to some embodiments.

FIGS. 17A-17B are side views of the cart of FIGS. 16A-16C, taken along A-A, according to some embodiments.

FIGS. 18A-18C illustrates perspective view of a light display system according to another embodiment.

FIGS. 19A-19C illustrate a light display system according to some embodiments.

FIG. 20 is a flowchart illustrating a method of treatment according to some embodiments.

### DETAILED DESCRIPTION

Before any embodiments of the application are explained in detail, it is to be understood that the application is not limited in its application to the details of construction and the arrangement of components set forth in the following description or illustrated in the following drawings. The application is capable of other embodiments and of being practiced or of being carried out in various ways.

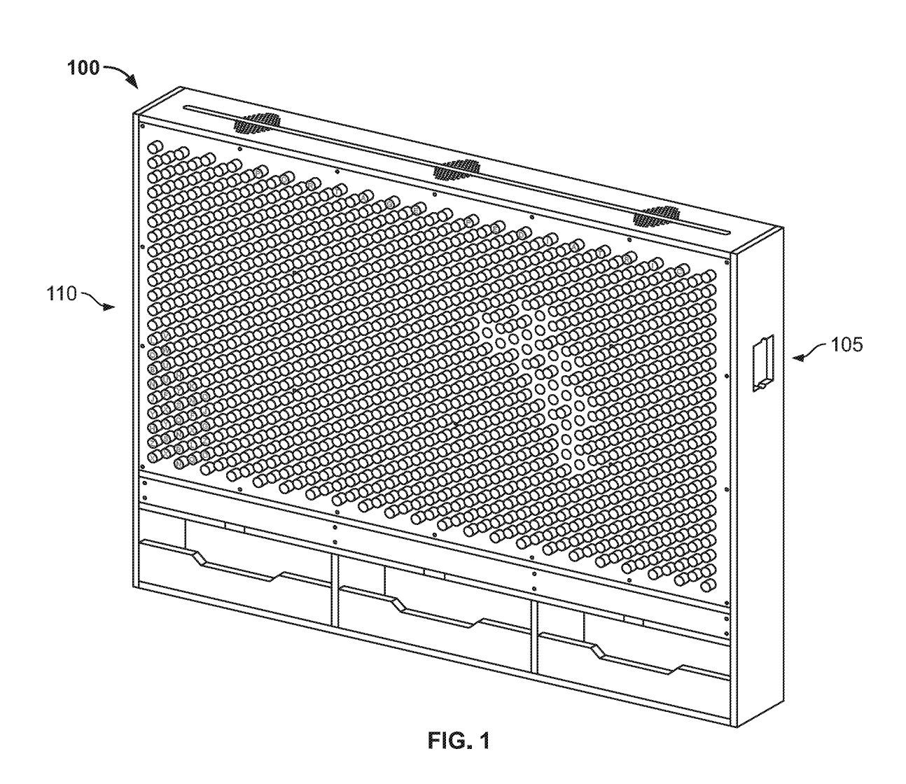

FIG. 1 is a front perspective view of a light display system 100 according to some embodiments. The light display system includes a rear portion 105 and a front portion 110. Although illustrated as a single unit, in some embodiments, the system 100 may be formed by a plurality of modular units.

As described in more detail below, the light display system 100 is an interactive light display configured to, among other things, be used: (1) to treat neurological disorders (including but not limited to, Alzheimer's, dementia, and autism); (2) for educational purposes (for example, spelling, mathematics, etc.); (3) for brand activation; and (4) for team building.

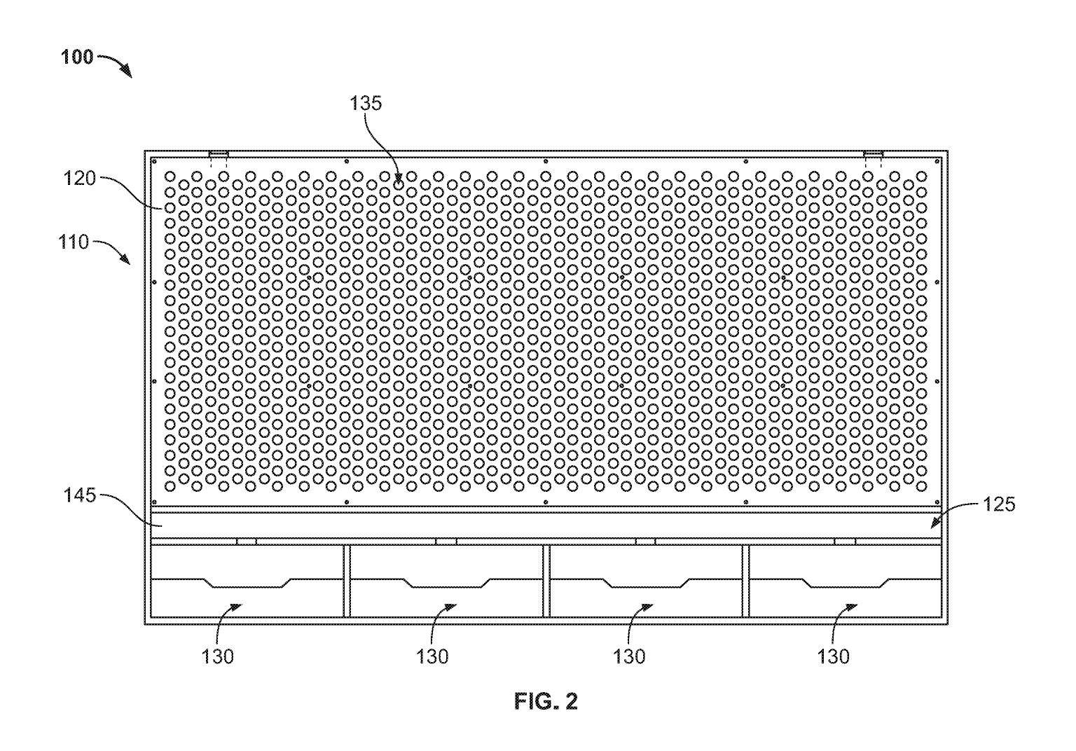

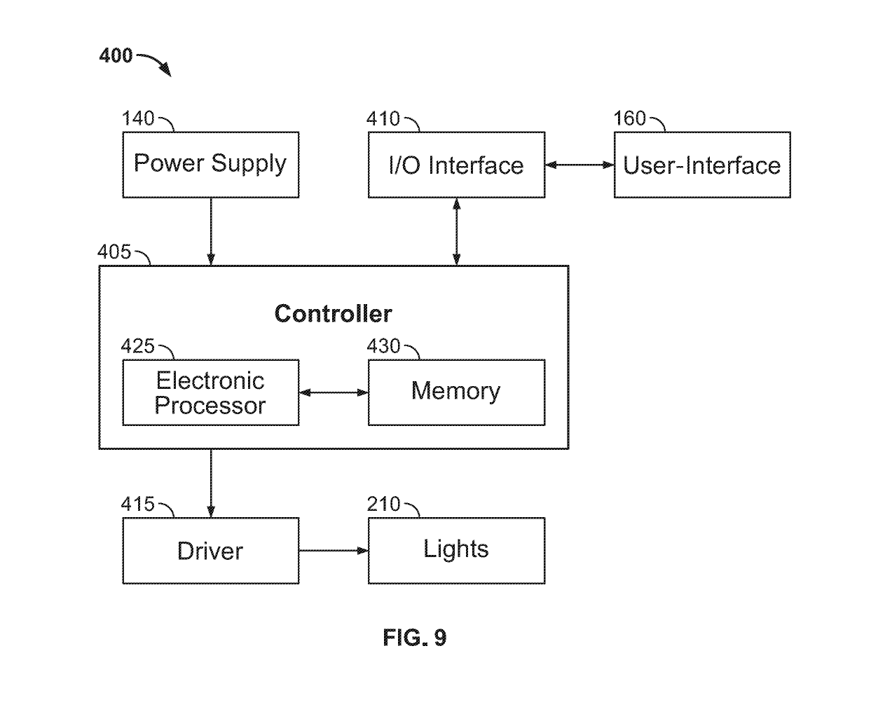

FIGS. 2-4 illustrate various perspectives of the light display system 100 according to some embodiments. The rear portion 105 includes a rear panel 115 (FIG. 7). The front portion 110 includes a front panel 120, a power supply compartment 125, and one or more storage compartments 130. In some embodiments, the rear portion 105 is formed at least partially of HDPE, fiber resin, wood, and/or metal. In some embodiments, the front portion 110 is formed, at least partially, of HDPE and/or fiber resin. In some embodiments, one or more exposed portions of the system 100 are formed of a surgical-grade material.

As discussed in more detail below, the front panel 120 includes a plurality of apertures 135. The front panel 120 may be formed of a polymer or similar materials. In some embodiments, the front panel 120 is a single unit.

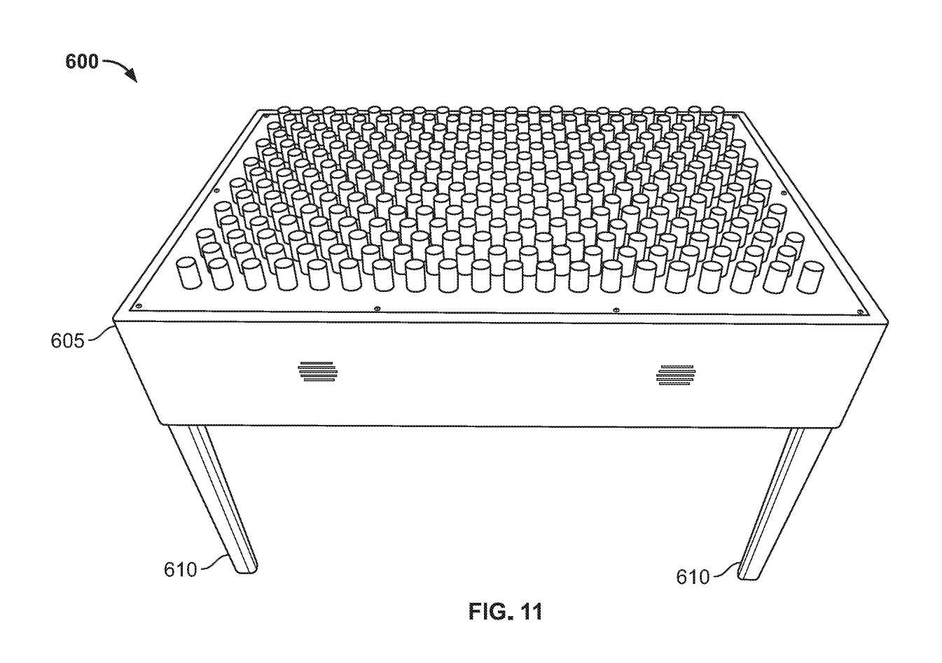

The power supply compartment 125 is configured to receive a power supply 140 (FIG. 9), as well as other control circuitry of the light display system 100. The power supply compartment 125 is enclosed by a power supply compartment faceplate 145. The power supply compartment faceplate 145 is located on the front portion 110 of the light display system 100 and is configured to be releasably attachable to the front portion 110. Such a configuration allows a user to access the power supply 140, as well as other control circuitry of the light display system 100, without requiring access to the rear portion 105 of the light display system 100. For example, in embodiments of the light display system 100 that are configured to be coupled to a wall, the power supply compartment faceplate 145 allows access to the power supply 140 and other control circuitry of the light display system 100 without removing the system 100 from the wall. As another example, in embodiments of the light display system 100 that are supported in a horizontal manner, the power supply compartment faceplate 145 allows access to the power supply 140 and other control circuitry of the light display system 100 without the need to overturn the system 100. In some embodiments, rear panel 115 may include one or more apertures at various locations for a power supply cord of the power supply 140.

The one or more storage compartments 130 are configured to receive one or more accessories (for example, one or more pegs 150 (FIG. 7), one or more game boards 155 (Fig. X), etc.). In some embodiments, the one or more storage compartments 130 are illuminated via a light source 132 (for example, one or more light-emitting diodes (LEDs), one or more light ropes, etc.) (illustrated in FIG. 7).

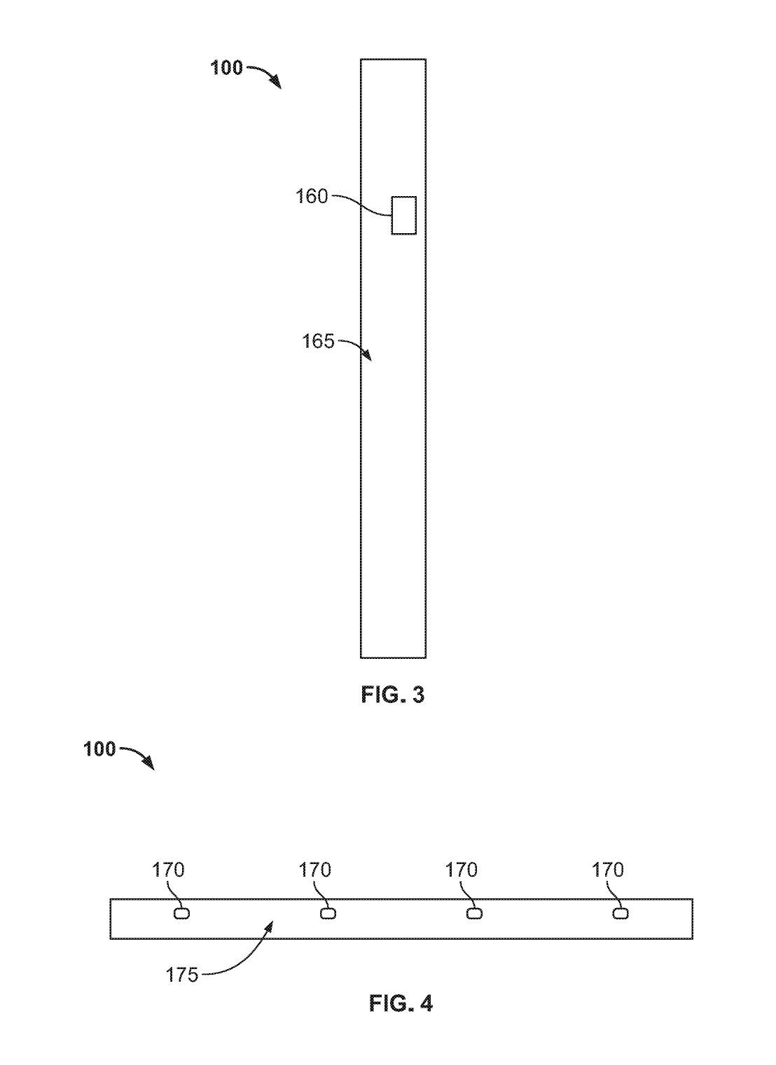

Although illustrated as being located on the front portion 110, and below the front panel 120, of the light system 100, in other embodiments the one or more storage compartments 130 may be located in other areas, including but not limited to, a side portion 165 (FIG. 3) and/or remote from the system 100. Additionally, although illustrated as having four storage compartments 130, in other embodiments, the system 100 may include less or more storage compartments 130.

The light display system 100 may further include a user-interface 160. In some embodiments, the user-interface 160 is located on the side portion 165 of the light display system 100. As described in more detail below, the user interface 160 is configured to power the light display system 100 on/off, as well as control the light display system 100 (for example, dimming the light display system 100). In other embodiments, the user-interface 160 may be located in other areas, including but not limited to, the front portion 110 and/or remote from the system 100.

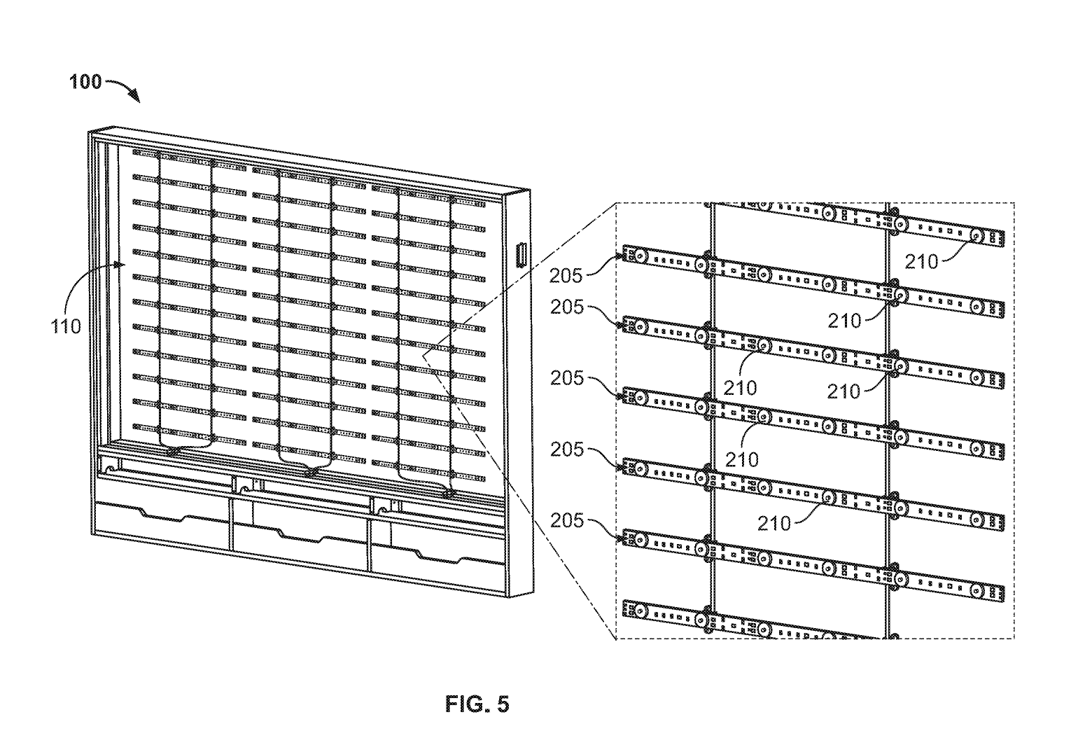

As illustrated in FIG. 4, the light display system 100 may further include one or more air holes 170 located on a tope portion 175. The air holes 170 are configured to dissipate heat from inside the system 100. In some embodiments, the light display system 100 includes one or more air holes 170 in other areas of the system, including but not limited to, the rear portion 105, the front portion 110, and one or more side portions 165.

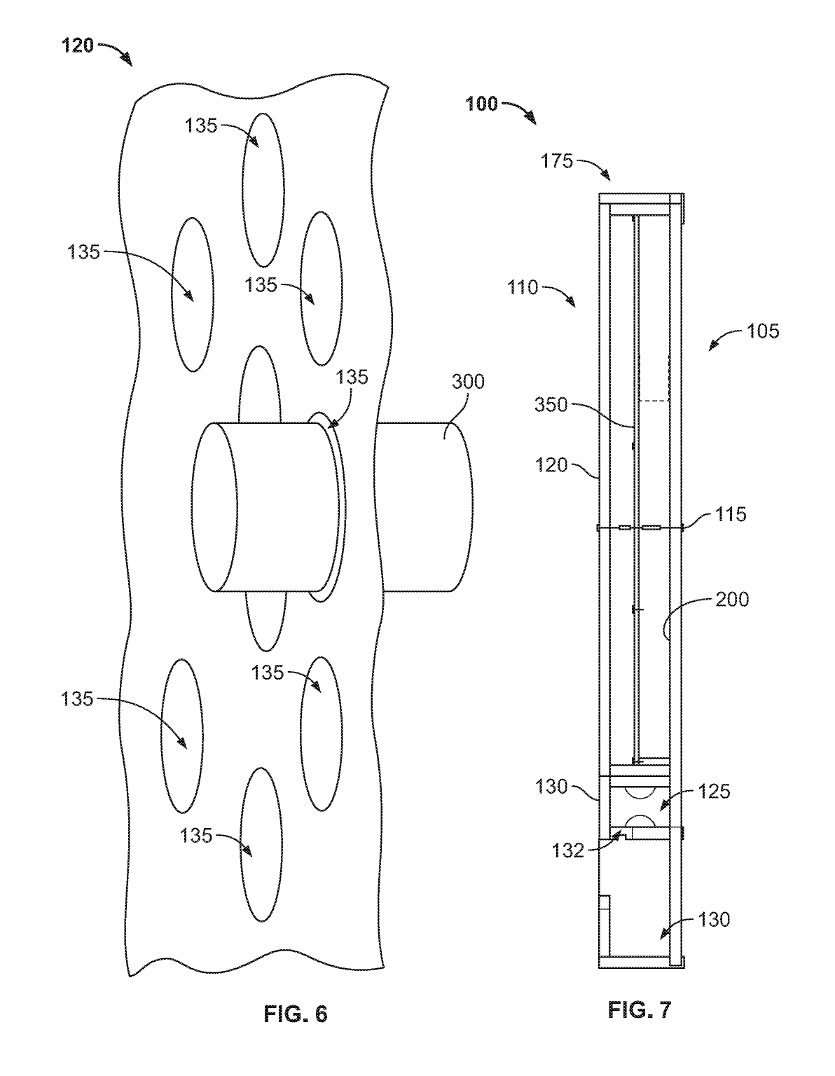

FIG. 5 is a perspective view of the light display system 100 according to some embodiments with the front panel 120 removed for illustrative purposes. As illustrated, the light display system 100 includes an illumination device 200. The illumination device 200 may include one or more illumination panels 205 each containing one or more lights 210. In some embodiments, the lights 210 are light-emitting diodes (LEDs). In other embodiments, the lights 210 are florescent lights. The illumination panels 205, along with the lights 210, are spaced apart from one another in order to provide consistent illumination of the light display system 100.

In some embodiments, the lights 210 are white lights. In other embodiments, the lights 210 are red, green, and blue (RGB) lights (for example, RGB LEDs). In yet other embodiments, the lights 210 are red, green, blue, and white (RGBW) lights (for example, RGBW LEDs). As discussed in further details below, the lights 210 may be configured to be controlled in a variety of manners including, but not limited to, dimming operations, varying color operations, blinking operations, steady operations, and patterned operations.

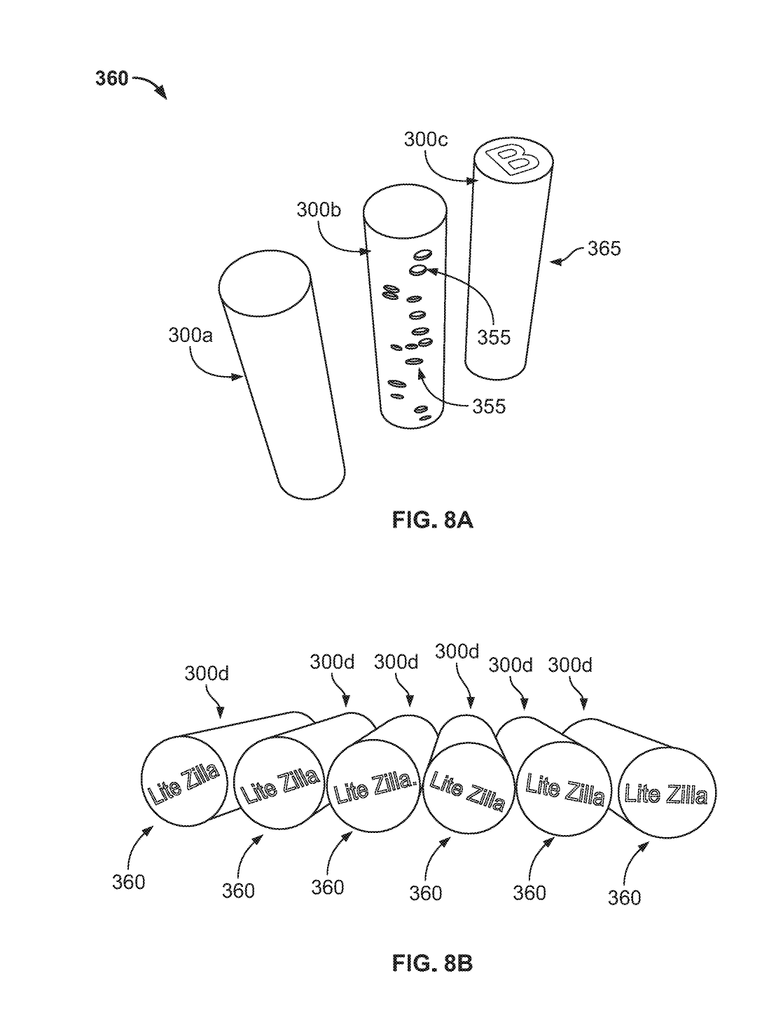

FIG. 6 illustrates an enlarged, cutaway view of the front panel 120 and plurality of apertures 135 according to some embodiments. The plurality of apertures 135 are each configured to receive a peg, or pin, 300. The pegs 300 are configured to be placed within an aperture 135 by a user. In some embodiments, when placed within an aperture 135, the pegs 300 are configured to protrude from the front panel 120 by approximately 1.5 inches to approximately 2 inches (for example, approximately 1.75 inches).

As illustrated in FIGS. 1, 2, and 5, the plurality of apertures 135 may be spaced in a predetermined pattern (for example, a staggered pattern). In some embodiments, the apertures 135 are spaced approximately one inch or less from each other. In some embodiments, the apertures 135 have a diameter of approximately one inch. In some embodiments, apertures 135 are configured to form an image (for example, a logo), rather than a rectangular grid.

FIG. 7 is a cutaway side view of the light display system 100 according to some embodiments. The illumination device 200 is coupled to the rear panel 115. A diffuser 350 is positioned a predetermined distance (for example, approximately two inches to approximately three inches) from the illumination device 200. The front panel 120, including the apertures 135, are then spaced a predetermined distance (for example, approximately one inch to approximately two inches) from the diffuser 350. The diffuser 350 is configured to diffuse or spread light from the illumination device 200 in order to promote consistent illumination of the light display system 100.

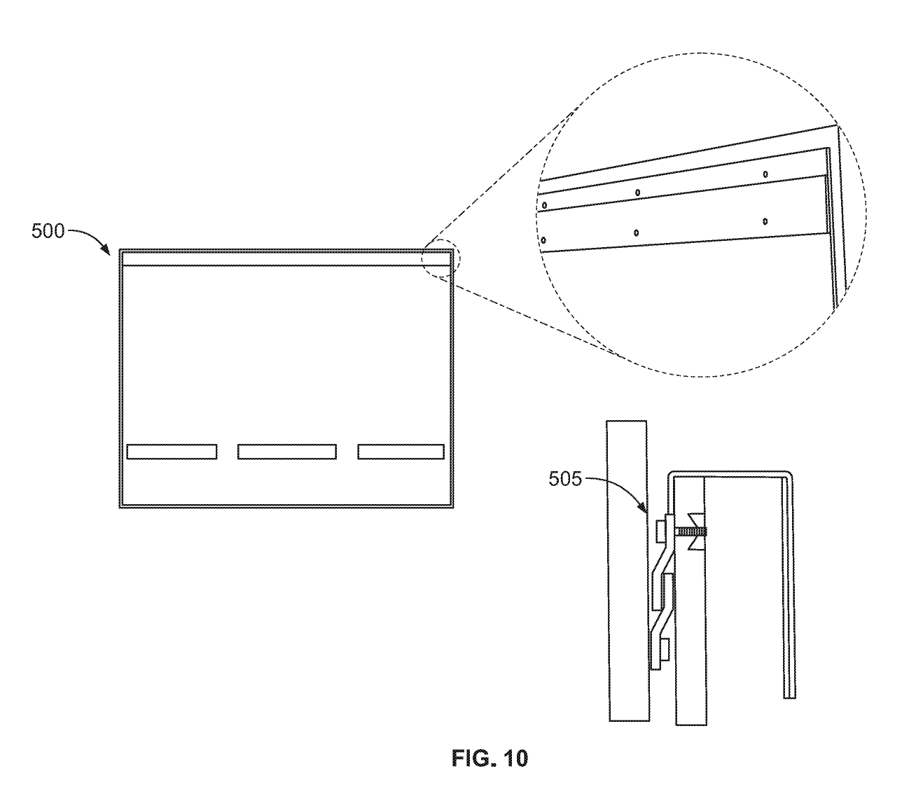

FIGS. 8A & 8B illustrate pegs 300a, 300b, 300c, and 300d according to some embodiments. Although illustrated as having a substantially cylindrical shape, in other embodiments, the pegs 300 may have various other shapes, including but not limited to, rectangular, triangular, and polygonal. In some embodiments, the pegs 300 are formed of an acrylic or similar material. In other embodiments, the pegs 300 are formed of glass or similar material. The pegs 300 may have a length of approximately 3.5 inches to approximately 4.5 inches (for example, 4 inches). In some embodiments, the pegs 300 having a diameter (for example, approximately one-half inch or greater) and/or have a length so as to prevent choking. As illustrated, peg 300a may be translucent and have a uniform color. Additionally, as illustrated, peg 300b may be translucent and include one or more bubbles 355. Pegs 300a, 300b may have a transparency rate of approximately 90% to approximately 95% (for example, approximately 93%).

As further illustrated in FIG. 8A, peg 300c may be opaque. In the illustrated embodiment, peg 300c further includes a mark 360. Although illustrated as being located on a top and/or bottom of peg 300c, mark 360 may be located on a side 365 of a peg 300. In some embodiments, mark 360 is a letter and/or symbol (for example, a symbol indicative of a chess piece and/or other board game piece). In operation, pegs 300a, 300b may be illuminated by light from the illumination device 200 when placed in an aperture 135, while peg 300c may block light from the illumination device 200 when placed in an aperture 135.

As illustrated in FIG. 8B, one or more pegs 300d may be any color and/or opaqueness. Additionally, pegs 800d may include a mark 360 in the form of a logo (for example, but not limited to a company logo, a company name, etc.). Similar to pegs 300a, 300b, pegs 300d may be illuminated by light from the illumination device 200 when placed in an aperture 135, or similar to peg 300c may block light from the illumination device 200 when placed in an aperture 135.

FIG. 9 is a block diagram of a control system 400 according to some embodiments. The control system 400 is configured to provide power and control of the light display system 100. The control system 400 includes a controller 405 that is electrically and/or communicatively connected to a variety of modules or components of the system 100. For example, the controller 405 is electrically and/or communicatively connected to the power supply 140, an input/output interface 410, and a driver 415. In some embodiments, the controller 405 is a DMX controller, or the like.

In some embodiments, the controller 405 includes a plurality of electrical and electronic components that provide power, operational control, and protection to the components and modules within the controller 405 and/or the system 100. For example, the controller 405 includes, among other things, an electronic processor 420 (for example, a microprocessor or another suitable programmable device) and the memory 425.

The memory 425 includes, for example, a program storage area and a data storage area. The program storage area and the data storage area can include combinations of different types of memory, such as read-only memory (ROM), random access memory (RAM). Various non-transitory computer readable media, for example, magnetic, optical, physical, or electronic memory may be used. The electronic processor 420 is communicatively coupled to the memory 425 and executes software instructions that are stored in the memory 425, or stored on another non-transitory computer readable medium such as another memory or a disc. The software may include one or more applications, program data, filters, rules, one or more program modules, and other executable instructions.

Power supply 140 is configured to supply nominal power to the controller 405 and/or other components of the system 100. In some embodiments, power supply 140 receives power from an external source (for example, an alternating-current (AC) power source). In other embodiments, the power supply 140 receives power from other power sources, for example a battery.

The I/O interface 410 provides an interface between system 100 and external devices (for example, the user-interface 160, a smartphone, a tablet, etc.). Depending on the particular application, the I/O interface 410 may support wired communications, wireless communications, or both. For example, the user-interface 160 may be physically wired to the I/O interface 410. As another example, the I/O interface 410 may be connected to an external device (for example, a wireless user-interface 160, a smartphone, a tablet, etc.) via a wireless connection (for example, a Bluetooth connection, a WiFi connection, a radio-frequency (RF) connection, etc.)

The driver, or driver module, 415 generates drive signals for the illumination device 200. For example, the driver 415 generates drive signals for one or more LEDs of the illumination device 200 based on specified or determined levels for the output currents provided to the LEDs (light intensity), a dimming level, fade-in times, fade-out times, etc.

In operation, controller 405 receives one or more user-control signals from an external device (for example, user-interface 160, smartphone, tablet, etc.). The controller 405 controls, via the driver 415, the lights 210 of the illumination device 200 to illuminate the light display system 100, in accordance with the one or more user-control signals.

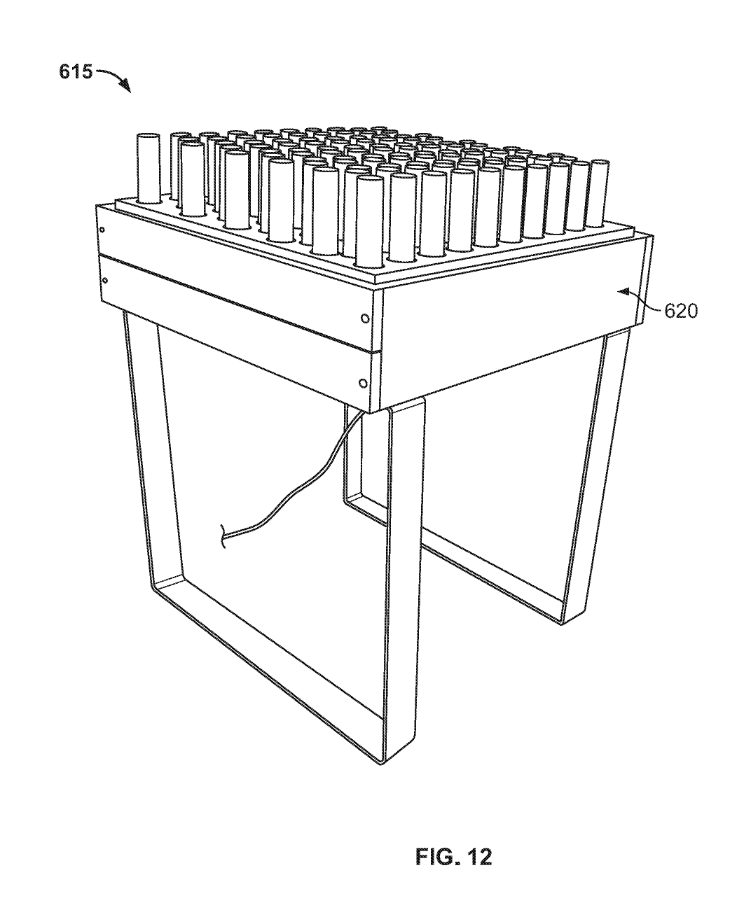

FIG. 10 illustrates a light display system 500 according to another embodiment. Light display system 500 includes similar components as light display system 100 discussed above. As illustrated, light display system 500 may be configured to be installed on a wall. As illustrated, light display system 500 may include a mounting system 505. In some embodiments, mounting system 505 is a two-part mounting system, such as but not limited to, a Z-clip mounting system and/or a French cleat mounting system.

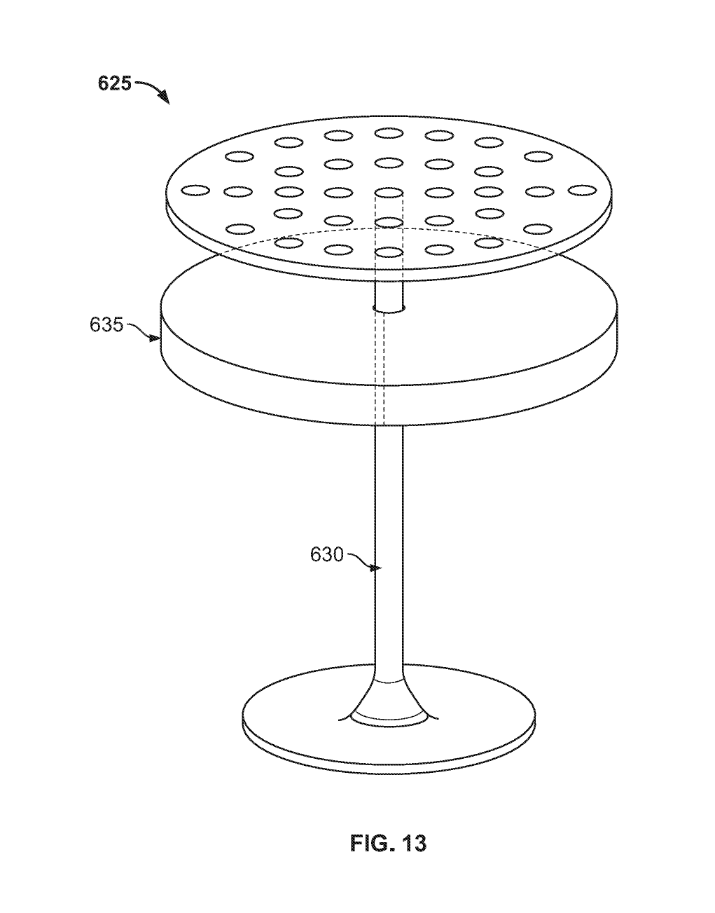

FIG. 11 illustrates a light display system 600 according to another embodiment. Light display system 600 may include similar components as light display system 100 discussed above. As illustrated, light display system 500 is a table-top version having a base 605 and one or more supports 610. In some embodiments, support 610 may include a wheel, or other rotational component, configured to provide movement of the system 600. Light display system 600 is configured to be in a horizontal orientation.

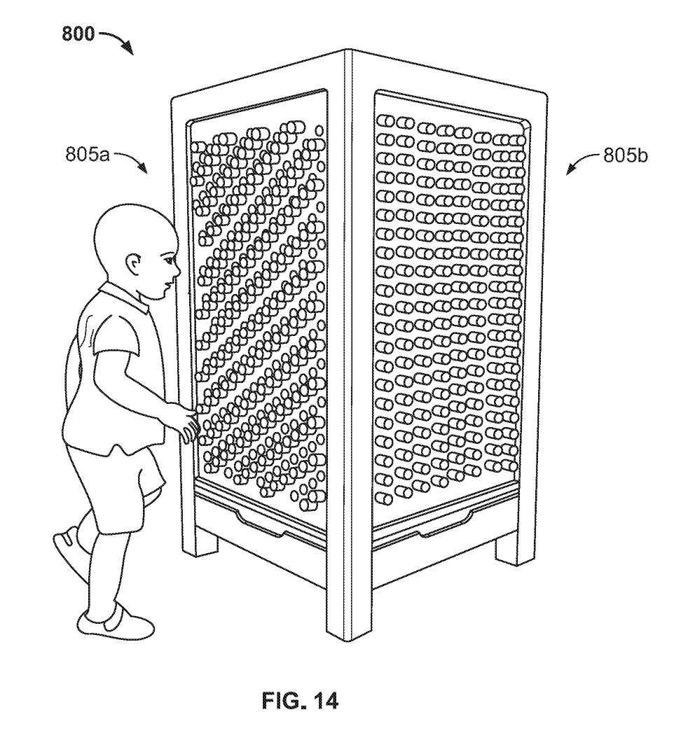

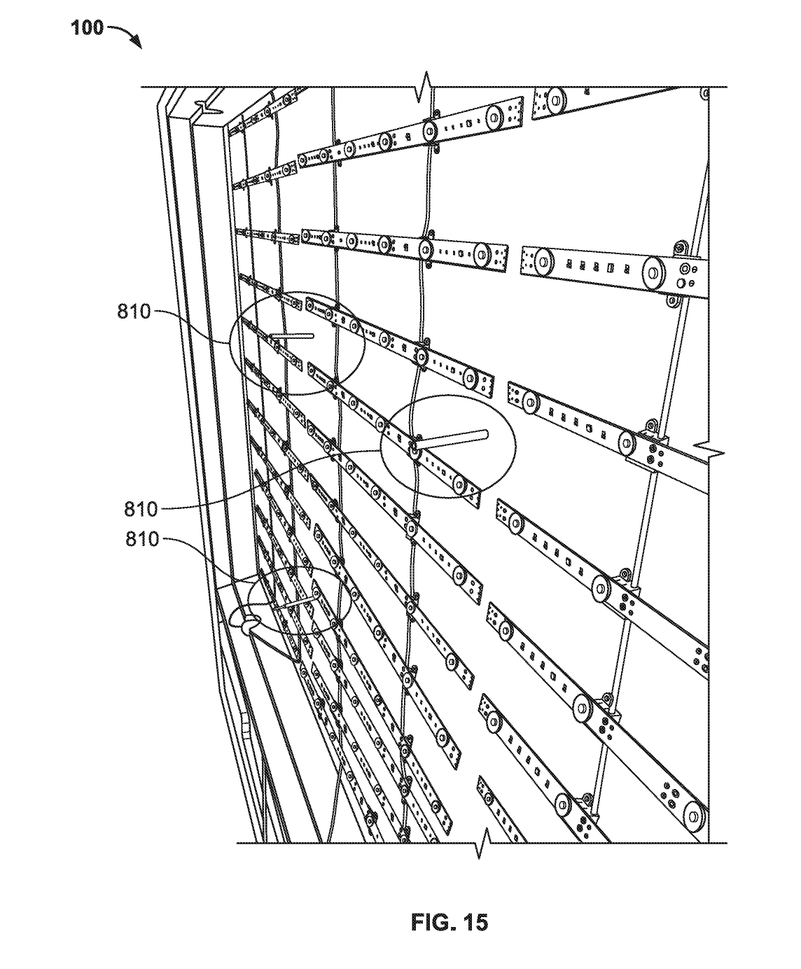

FIG. 12 illustrates a light display system 615 according to another embodiment. Light display system 615 may include similar components as light display system 100 discussed above. As illustrated, in some embodiment, light display system 615 includes one or more drawers, or cubbies, 620 for storage purposes. FIG. 13 illustrates a light display system 625 according to another embodiment. Light display system 625 may include similar components as light display system 100 discussed above. As illustrated, light display system 625 includes a support 630 configured to support a base 635. In some embodiments, support 630 has an adjustable length configured to adjust the height of the base 635, and thus the overall height of the light display system 625.

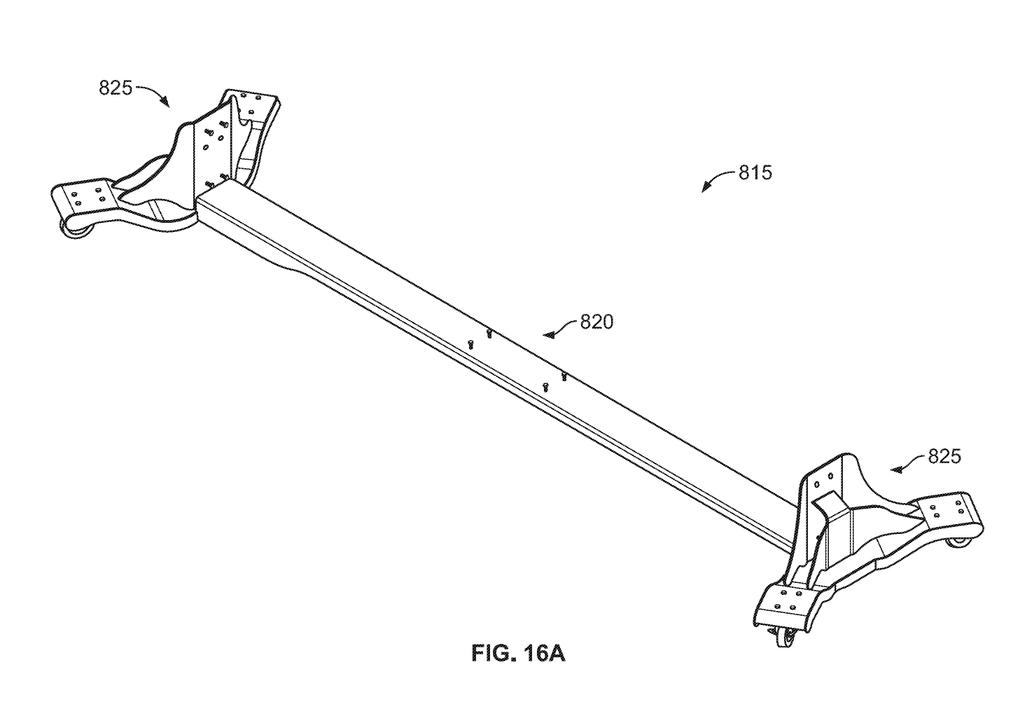

FIG. 14 illustrates a light display system 800 according to another embodiment. Light display system 800 may include similar components as light display system 100 discussed above. Light display system 800 includes two or more sides 805a, 805b, each having a front panel 120. Although not illustrated, light display system 800 may include additional sides 805 having additional front covers 120. For example, in some embodiments the light display system 800 includes three sides 805. In yet another embodiment, the light display system 800 includes four sides 805. In some embodiments, the system 800 may further include a top, or cap. In such an embodiment, the top may have a triangular, or pyramid, shape in order for the light display system 800 to resemble a building or skyscraper.

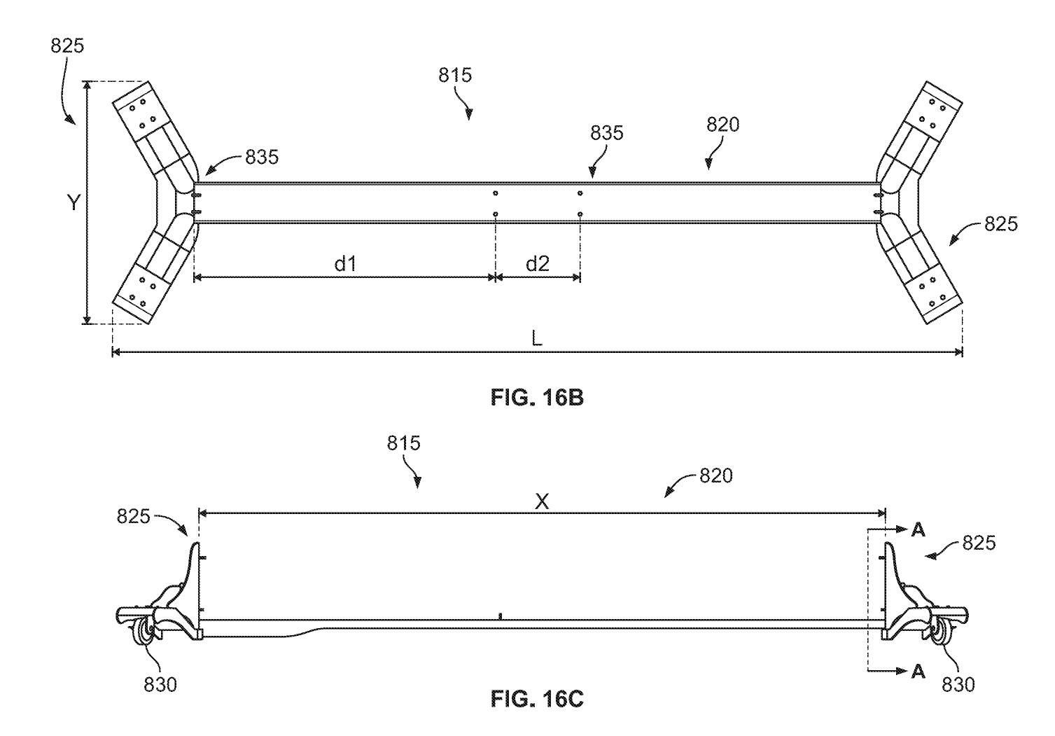

FIG. 15 is a perspective view of the light display system 100 according to some embodiments with the front panel 120 removed for illustrative purposes. As illustrated, the light display system 100 may further include one or more supports 810. The one or more supports 810 are configured to support the illumination device 200, the diffuser 350, and or the front cover 120. Furthermore, the supports 810 provide support to the light display system 100.

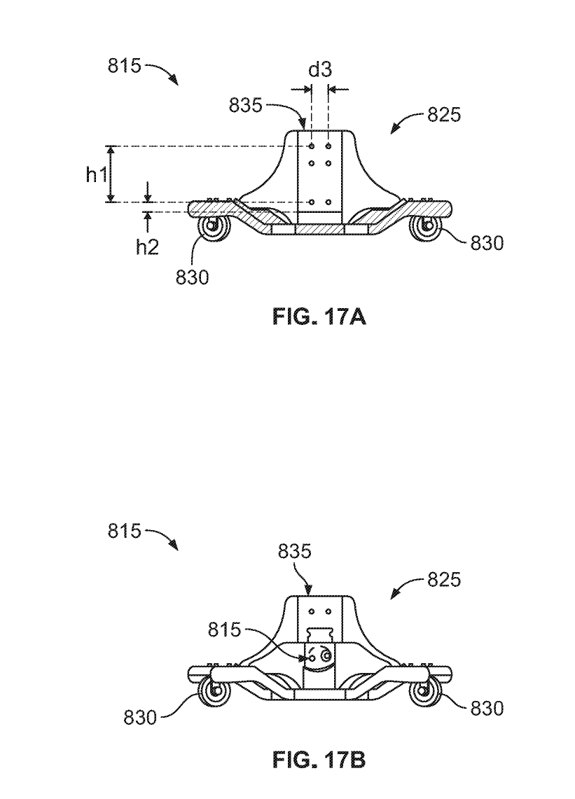

FIGS. 16A-16C & 17A-17B illustrate a cart 815 of a light display system 100 according to some embodiments. The cart 815 may be configured to support the light display system 100, as well as provide movement of the light display system 100. As illustrated, the cart 815 may include a base 820 configured to support light display system 100. The base 820 may be supported by one or more side portions 825, each having one or more wheels 830.

As illustrated, the cart 815 may have an overall length L, while base 820 has a length X, and the side portions 825 have side lengths Y. In some embodiments, the length L, length Y, and side lengths Y are determined based on the geometry (for example, height, length, and depth) of the light display system 100.

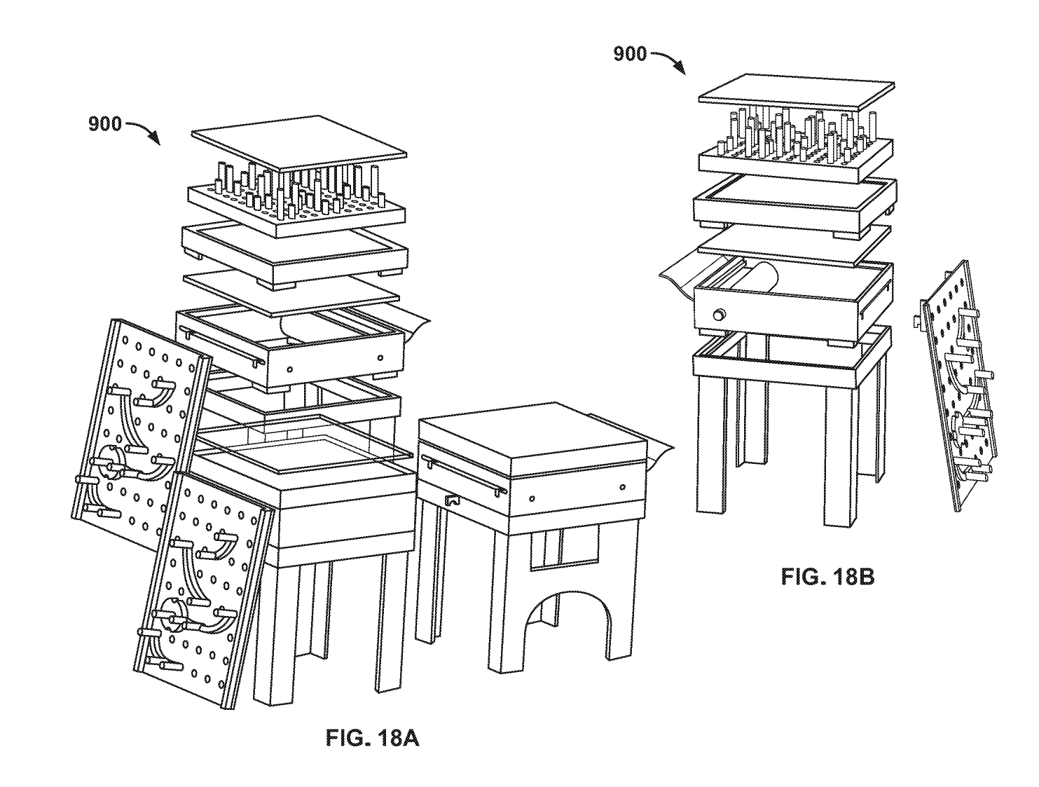

In the illustrated embodiments, the cart 815 includes one or more apertures 835 configured to secure the system 100 to the cart. The one or more apertures 835 may be located in various positions along the base 820 and/or the side portions 825. As illustrated in FIG. 16B, one or more apertures 825 may be located at various distances d1, d2 of the base 820 and/or at various heights h1, h2 and/or distances d3 of the side portions 825. As illustrated in FIG. 17B, one or more of the side portions 825 may further include a brake 840 configured to prohibit movement of the cart 815.

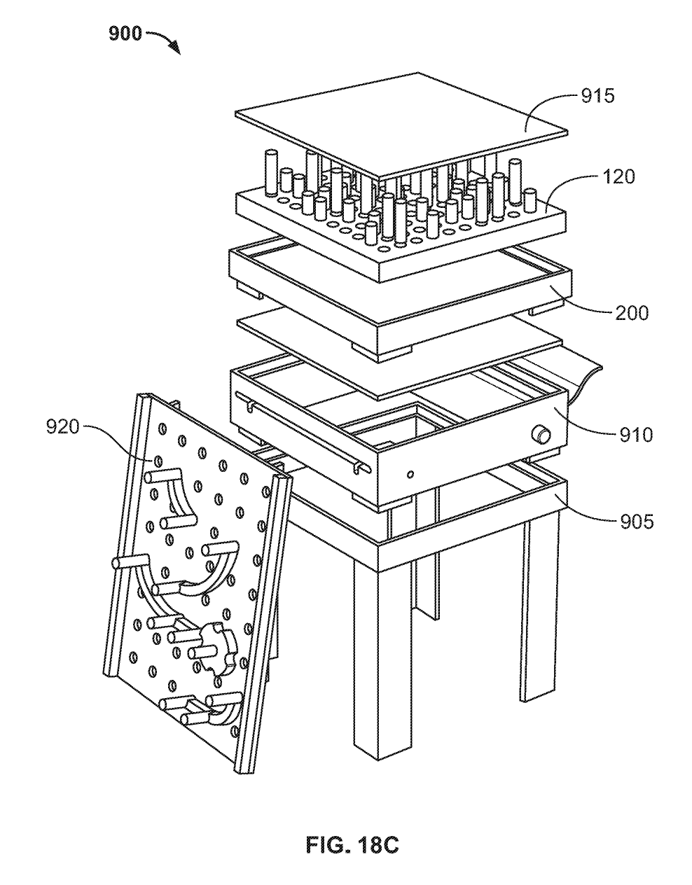

FIGS. 18A-18C illustrate various views of a light display system 900 according to another embodiment. Light display system 900 may include similar components as light display system 100 discussed above. Light display system 900 may further include one or more modules, for example, a base 905, an art module 910, an illumination device 200, a front cover 120, a table top 915, and a peg board 920. The base 905 is configured to support the system 900. The art module 910 may be configured to support art supplies (for example, a roll of paper). The table top 915 is configured to be placed above the front cover 120 and provide a table-like surface to a user. The peg board 920 includes a plurality of apertures (for example, apertures similar to apertures 135). The peg board 920 may be configured to receive pegs 300, or other peg-like devices. As discussed above, in some embodiments, pegs 300 include one or more marks 360 (for example, a mark indicative of a game piece, such as but not limited to a chess piece).

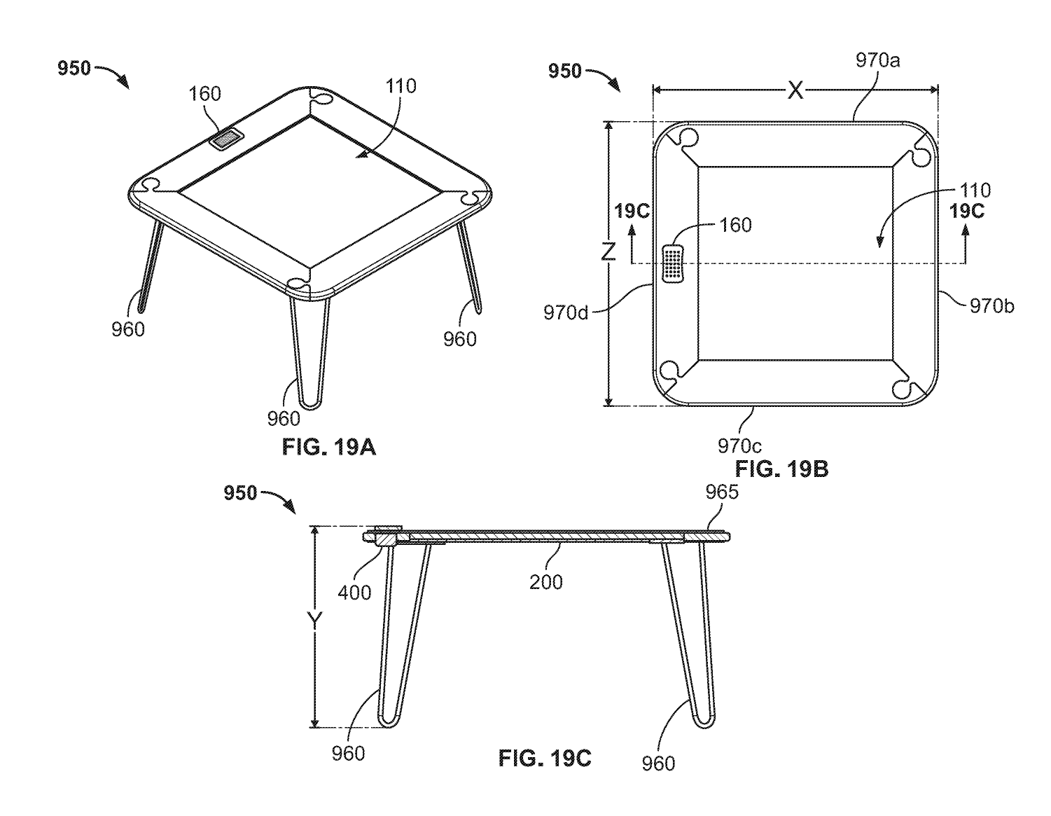

FIGS. 19A-19C illustrate various views of a light display system 950 according to another embodiment. Light display system 950 may include similar components as light display system 100 discussed above. Light display system 950 may further include top, or table top, 955, one or more legs 960, front portion 110, and the user-interface 160. The light display system 950 may further include a bezel 965 surrounding the front portion 110. In the illustrated embodiment, the bezel 965 is formed of a plurality of bezel pieces 970a-970d coupled together (for example, via tabs (or tongues) and blanks). In other embodiments, the bezel 965 is formed of a single piece.

The disclosed embodiments may be incorporated into the treatment of a subject (such as a patient, which may be a human or non-human). For example, the disclosed embodiments may be used in methods for treatment of disorders, such as neurological, cognitive, and/or psychiatric disorders. In some embodiments, the disorders include dementia (associated with Alzheimer's disease, ischemia, multi-infarct dementia, trauma, vascular problems or stroke, HIV disease, Parkinson's disease, Huntington's disease, Pick's disease, Creutzfeldt-Jacob disease, perinatal hypoxia, other general medical conditions or substance abuse), dementia due to HIV disease, dementia due to Huntington's disease, dementia due to Parkinson's disease, Parkinsonian-ALS demential complex, dementia of the Alzheimer's type, age-related cognitive decline, mild cognitive impairment, autism, and/or insomnia.

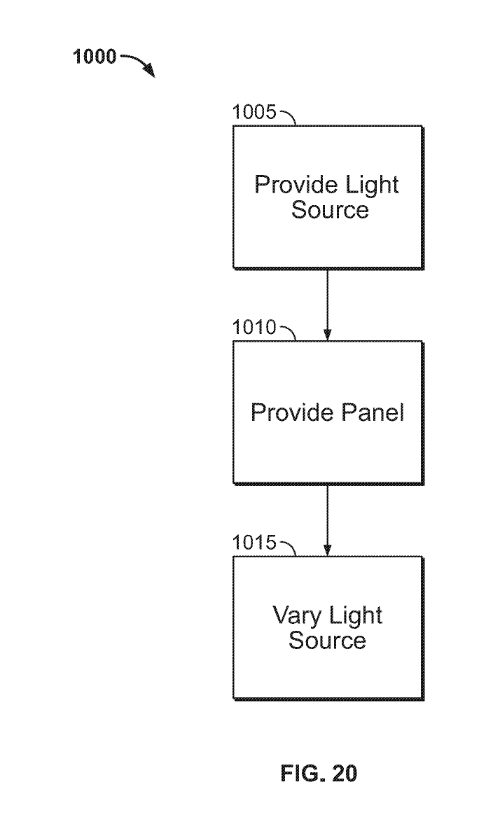

FIG. 20 is a method, or operation, of treatment 1000 according to some embodiments. Method of treatment 1000 includes providing a light source directed toward a subject (block 1005). In some embodiments, the light source provided toward the subject may be approximately 2,000 lux to approximately 10,000 lux.

Providing a panel between the patient and the light source (block 1010). The panel (for example, front panel 120) may include a plurality of apertures (for example, aperture 135). Each aperture may be configured to receive a peg (for example, peg 300) placed by the subject. Once placed in the aperture, the peg may be illuminated by the light source. The light source may then be varied (block 1015). In some embodiments, varying of the light source includes varying the color, varying the brightness, and varying the intensity.

Treatment 1000 may reward functions in the subject that can positively impact mood, perceptions of self, and behavior. Furthermore, a non-verbal subject may express oneself through use of the disclosed embodiments.

Treatment 1000 may further influence the circadian rhythm of the subject to improve sleep-wake cycles and sleep quality, while decreasing sundown syndrome. The circadian rhythm of sleep and wakefulness, as well as sundowning behavior, may be controlled by the suprachiasmatic nucleus and the daily rhythm of melatonin secretion. Low melatonin levels stimulate wakefulness, and increasing melatonin levels induces drowsiness and sleep. While the suprachiasmatic deteriorates in subjects with dementia, melatonin levels can be strongly influenced by exposure to a light source. Treatment 1000 may provide light (for example, blue spectrum light) to a subject to effectively maintain circadian rhythm and a higher intensity of daytime light than normal daily activity exposure.

Visuospatial dysfunctions may be an early manifestation of Alzheimer's disease, and is also present in subjects with vascular dementia, Lewy body disease, and Parkinson's disease. Potential causes of visuospatial disorders include dysfunction in multiple visual capacities (for example, depth perception, color discrimination, and contrast sensitivity), visual inattention, and unilateral or bilateral visual neglect. Treatment 1000 may improve spatial function by providing the subject with pegs of varying colors to be placed in one or more apertures exposed to the light source.

Thus, the application provides, among other things, a light display system and method for treating a disorder with use of the light display system. Various features and advantages of the application are set forth in the following claims.

What is claimed is:

1. A light display system comprising:
a rear panel;
a light source supported by the rear panel, the light source configured to output light;
a diffuser having a first side and a second side, the first side proximate the light source, the diffuser configured to diffuse the light outputted by the light source;
a front panel located proximate the second side of the diffuser, the front panel including a plurality of apertures, each aperture configured to receive a peg; and
a power supply configured to provide power to the light display.

2. The light display system of claim 1, wherein the plurality of apertures are spaced in a staggered positioning.

3. The light display system of claim 1, wherein the light source is a red, green, blue, white (RGBW) light-emitting diode (LED) source.

4. The light display system of claim 1, further comprising a compartment.

5. The light display system of claim 4, wherein the compartment is located at a bottom portion of the light display system.

6. The light display system of claim 4, wherein the compartment is located at at least one selected from a group consisting of a side of the light display system and remote from the light display system.

7. The light display system of claim 4, wherein the compartment is illuminated via a backlight.

8. The light display system of claim 1, further comprising:
a power supply compartment configured to receive the power supply, and
a power supply compartment faceplate configured to enclose the power supply within the power supply compartment,
wherein the power supply compartment faceplate is configured to secure to a front portion of the light display system.

9. The light display system of claim 1, wherein the front panel is a single unit.

10. The light display system of claim 1, wherein the peg is translucent.

11. The light display system of claim 7, wherein the peg includes a plurality of bubbles.

12. The light display system of claim 1, wherein the peg is opaque.

13. The light display system of claim 1, wherein the peg includes a translucent portion and an opaque portion.

14. The light display system of claim 1, further comprising a support configured to support the light display system in a horizontal configuration.

15. The light display system of claim 11, wherein the support includes a wheel.

16. The light display system of claim 1, wherein the front panel includes a game board.

17. The light display system of claim 13, wherein the pegs include at least one selected from a group consisting of a bubble and a marking.

18. The light display system of claim 1, further comprising a controller having an electronic processor and a memory, the controller configured to:
receive a user-control signal, and
vary the light source in response to the user-control signal.

19. A method of treating a neurological disorder in a subject, the method comprising:
providing a light source directed toward the patient;
providing a panel between the patient and the light source, the panel including a plurality of apertures, each aperture configured to receive a peg placed by the patient for treating the neurological disorder, wherein the peg is illuminated by the light source when placed in an aperture of the plurality of apertures; and
varying, via a controller, the light source directed toward the patient for treating the neurological disorder.

20. The method of claim 19, wherein the plurality of apertures are spaced in a staggered positioning.

21. The method of claim 19, wherein the light source is a red, green, blue, white (RGBW) light-emitting diode (LED) source.

22. The method of claim 19, wherein the light source is a red, green, and blue (RGB) light-emitting diode (LED) source.
